(19) United States
(12) Patent Application Publication
(10) Pub. No.: US 2026/0207159 A1
(43) Pub. Date: Jul. 23, 2026

(54) METHOD AND SYSTEM FOR ACQUIRING CT EQUIANGULAR DATA, STORAGE MEDIUM, AND CT SCANNING SYSTEM

(71) Applicant: IRAY GROUP, Shanghai (CN)

(72) Inventors: Jinling LIU, Solon, OH (US); Hongbin GUO, Solon, OH (US); Fei HU, Shanghai (CN)

(73) Assignee: IRAY GROUP, Shanghai (CN)

(21) Appl. No.: 19/452,449

(22) Filed: Jan. 19, 2026

(30) Foreign Application Priority Data
Jan. 17, 2025 (CN) 2025100771977

(51) Int. Cl. A61B 6/58; A61B 6/00; A61B 6/03

(52) U.S. Cl. CPC A61B 6/586; A61B 6/032; A61B 6/547

(57) ABSTRACT

A method and a system for acquiring CT equiangular data, a storage medium, and a CT scanning system are disclosed. The method comprises: acquiring offset errors of a rotor encoder of the CT scanning system; calculating compensation amounts for the rotor encoder based on the offset errors; applying each of the compensation amounts as a delay to a corresponding theoretical trigger signal of the data measurement system of the CT scanning system to generate a corrected trigger signal; and correcting data acquisition for a current view based on a trigger interval of a previous view of the CT scanning system. The disclosed method, system, storage medium, and CT scanning system achieve equiangular data acquisition of the CT scanning system through rotor encoder error compensation and rotor speed error compensation.

### FIELD OF THE INVENTION

The present disclosure relates to the technical field of Computed Tomography (CT) scanning and imaging. More particularly, the present disclosure pertains to a method and a system for acquiring CT equiangular data, a storage medium, and a CT scanning system.

### BACKGROUND OF THE INVENTION

CT is a widely used medical imaging device. A typical CT scanner comprises a stationary portion (the stator) and a rotating portion (the rotor). The rotor section mainly comprises two major systems, generally referred to as the X-ray generation system and the detector system (so-called Data Measurement System-DMS). The stator supports the rotor through bearings. A slip ring is generally disposed between the stator and rotor to supply power, and provide command and/or data communication channels. A patient table supports the patient and transports the patient while the scanner stays stationary. In some implementations, the patient table remains stationary while the stator and rotor move relative to the patient table.

During CT scanning, data acquisition is typically performed at equal angular intervals. That is, the DMS reads one view of data at each predefined, evenly spaced rotational angle of the rotor. FIG. 1 illustrates an example of rotor angle signal transmission and processing. As shown in FIG. 1, a rotor encoder outputs A pulses and B pulses that represent the rotational angle, while the phase difference between the A pulses and the B pulses indicates the direction of rotation.

An Index Pulse (IP) provides a reference rotational position. Either the A pulses or B pulses may be used to measure rotor angular displacement, and using both pulses together increases angular resolution by a factor of four. In one implementation, the rotor encoder signals are processed by a Field-Programmable Gate Array (FPGA) on a stator-side printed circuit board assembly (PCBA), referred to as the stator control board (SCB). The processed signals are then transmitted to the rotor side, where another FPGA on a rotor-side PCBA, referred to as the rotor control board (RCB), receives and processes said processed signals. The RCB FPGA outputs trigger signals to the DMS so that data corresponding to each predefined angular rotation is acquired based on the rotor encoder signals. The RCB further controls the high-voltage generator (HVG) to ensure that the switching of the X-ray emission is synchronized with DMS data acquisition.

However, existing CT systems still face several challenges:
(1) Ideally, equiangular CT data acquisition requires that each acquisition trigger correspond to an equal rotational angle. However, encoder tolerances, mechanical imperfections, and variations in rotational speed cause the actual angular positions of acquisition triggers to deviate from uniform spacing. The varying integration time for each data acquisition leads to significant fluctuations and even errors in data from different perspectives, placing considerable pressure on CT reconstruction algorithms. In some designs, to ensure that the DMS (Digital Measurement System) completes the data measurement system integration before the integration signal arrives, the integration time is typically set to be less than the minimum possible angle time before each integration begins. This often results in premature integration termination, degrading the quality of the acquired signal. However, this only prevents data errors in the DMS and does not effectively address data fluctuations.
(2) The actual angular span covered during each DMS integration is inconsistent. Although the process is nominally described as equiangular acquisition, each view may correspond to a different angular coverage, thereby reducing the quality of the reconstructed image.
(3) Ensuring system performance requires stringent control over the rotational speed control system of the rotating gantry and the accuracy of the rotary encoder. This limits the use of lower-cost components, thereby increasing the overall system cost.

### SUMMARY OF THE INVENTION

The present disclosure provides a method and a system for acquiring CT equiangular data, a storage medium, and a CT scanning system, which achieve equiangular data acquisition of the CT scanning system through rotor encoder error compensation and rotor speed error compensation.

In a first aspect, the present disclosure provides a method for acquiring CT equiangular data applied to a CT scanning system. The method comprises acquiring offset errors of a rotor encoder of the CT scanning system; calculating compensation amounts for the rotor encoder based on the offset errors; applying each of the compensation amounts as a delay to a corresponding theoretical trigger signal of a data measurement system of the CT scanning system, to generate a corrected trigger signal; and correcting data acquisition for a current view based on the corrected trigger signal and a trigger interval of a previous view of the CT scanning system.

In one embodiment of the first aspect, acquiring the offset errors of the rotor encoder of the CT scanning system comprises:
calculating an offset error Error (i) corresponding to each pulse of the rotor encoder according to Error(i)=AngAct(i)−AngIdeal(i), wherein AngIdeal(i) represents a theoretical angle of an i-th pulse among the pulses, and AngAct(i) represents an actual angle of the i-th pulse;
determining a maximum pulse delay of the rotor encoder according to MaxDelay=Max(Error(1), Error(2), . . . , Error(N)), wherein N represents a total number of pulses generated by the rotor encoder for one full rotation, and 1≤i≤N; and
calculating the offset errors of the rotor encoder according to ErrorNew(i)=MaxDelay−Error(i)+SafMar, wherein SafMar represents a safety margin.

In one embodiment of the first aspect,

$$AngIdeal ( i ) = ( \frac{360}{N} ) * i .$$

In one embodiment of the first aspect, calculating the compensation amounts for the rotor encoder based on the offset errors comprises:

acquiring a rotation time RotTime corresponding to one full rotation of a rotor of the CT scanning system; and

calculating the compensation amounts according to Comp(i)=Round(ErrorNew(i)/(360*RotTime/ClockCycle), wherein ClockCycle represents a counter cycle of the CT scanning system for processing signals of the rotor encoder.

In one embodiment of the first aspect, a first pulse following an index pulse of the rotor encoder is configured as a zero-angle position of a rotor of the CT scanning system.

In one embodiment of the first aspect, the method further comprises: each time after receiving an index pulse of the rotor encoder, reacquiring the offset errors of the rotor encoder of the CT scanning system to update the corrected trigger signal.

In one embodiment of the first aspect, correcting the data acquisition for the current view based on the corrected trigger signal and the trigger interval of the previous view of the CT scanning system comprises:
acquiring the trigger interval Int (f) of the previous view (f) by subtracting an actual trigger time of a trigger signal of view (f−1) before the previous view (f) from an actual trigger time of a trigger signal of the previous view (f);
calculating a predicted trigger interval IntNew (f) for the current view (f+1) by subtracting a redundancy amount and/or an integration-termination advance amount from the trigger interval Int (f); and
performing the data acquisition for the current view (f+1) based on the corrected trigger signal and the predicted trigger interval.

In a second aspect, the present disclosure provides a system for acquiring CT equiangular data, applied to a CT scanning system. The system comprises a trigger error acquisition module, a calculation module, a compensation module, and a view data acquisition module.

The trigger error acquisition module is configured to acquire offset errors of a rotor encoder of the CT scanning system.

The calculation module is configured to calculate compensation amounts for the rotor encoder based on the offset errors.

The compensation module is configured to apply each of the compensation amounts as a delay to a corresponding theoretical trigger signal of a data measurement system of the CT scanning system, to generate a corrected trigger signal.

The view data acquisition module is configured to correct data acquisition for a current view based on the corrected trigger signal and a trigger interval of a previous view of the CT scanning system.

In a third aspect, the present disclosure provides a CT scanning system, comprising a processor and a memory.

The memory is configured to store a computer program.

The processor is configured to execute the computer program stored in the memory, to cause the CT scanning system to perform the method for acquiring CT equiangular data as described in any one of the embodiments provided in the first aspect of the present disclosure.

In a fourth aspect, the present disclosure provides a non-transitory computer-readable storage medium, which stores a computer program. The method for acquiring CT equiangular data as described in any one of the embodiments provided in the first aspect of the present disclosure is implemented when the computer program is executed by a processor.

As described above, the method and the system for acquiring CT equiangular data, the storage medium, and the CT scanning system of the present disclosure provide the following beneficial effects.
(1) Equiangular data acquisition of the CT scanning system is achieved through rotor encoder error compensation and rotor speed error compensation.
(2) The preset integration time of each view can be adjusted according to variations in rotor speed, thereby maximizing the angular coverage of data acquisition.
(3) High system stability and excellent image quality can be provided.
(4) Low-cost rotational speed control systems and rotor encoders may be used in CT systems, thereby reducing the overall cost of the CT system and enhancing practicality.

### BRIEF DESCRIPTION OF DRAWINGS

FIG. 1 illustrates a schematic diagram of transmission and processing of rotor angle signals in a CT scanning system in the prior art,

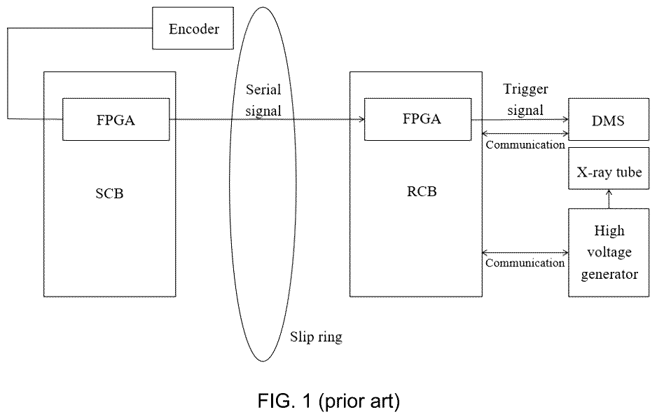

FIG. 2 illustrates a flowchart of a method for acquiring CT equiangular data in one embodiment of the present disclosure;

FIG. 3 illustrates a schematic diagram of error compensation of a rotor encoder in one embodiment of the present disclosure;

FIG. 4 illustrates a schematic diagram of trigger timing of a rotor in one embodiment of the present disclosure;

FIG. 5 illustrates a schematic diagram of rotor speed error compensation in one embodiment of the present disclosure;

FIG. 6 illustrates a block diagram of a system for acquiring CT equiangular data in one embodiment of the present disclosure; and

FIG. 7 illustrates a block diagram of a CT scanning system in one embodiment of the present disclosure.

### DETAILED DESCRIPTION OF THE INVENTION

The embodiments of the present disclosure will be described below. Those skilled can easily understand advantages and effects of the present disclosure according to contents disclosed by the specification. The present disclosure can also be implemented or applied through other different exemplary embodiments. Various modifications or changes can also be made to all details in the specification based on different points of view and applications without departing from the spirit of the present disclosure. It should be noted that the following embodiments and the features of the following embodiments can be combined with each other if no conflict will result.

It should be noted that the drawings provided in this disclosure only illustrate the basic concept of the present disclosure in a schematic way, so the drawings only show the components closely related to the present disclosure. The drawings are not necessarily drawn according to the number, shape, and size of the components in actual implementation; during the actual implementation, the type, quantity, and proportion of each component can be changed as needed, and the components' layout may also be more complicated.

During equiangular data acquisition of a CT scanner, variations in trigger signals of the DMS are primarily caused by rotor encoder errors and rotor speed variations.

The errors of the pulse signals of the rotor encoder mainly result from manufacturing tolerances and/or defects of the rotor encoder, such as slot position tolerances and/or slot size tolerances of the rotor encoder. The pulse positions corresponding to specific angular positions for each rotation are repeatable from rotation to rotation, whereas the intervals between pulses may change abruptly. When both A pulses and B pulses are used simultaneously to increase angular measurement resolution, phase errors between the A pulses and the B pulses constitute another source of trigger signal variation. These phase errors are also repeatable. In summary, both the pulse signals of the rotor encoder and the phase errors between the A pulses and the B pulses may change abruptly, but such changes are repeatable for each rotation.

In addition, because the rotor frame and the components mounted on the rotor frame have large inertia, variations in rotor speed can only occur gradually. Under a nominally constant rotor speed, actual rotor speed variations may be caused by fixed factors (such as mass distribution and imbalance of rotor components) and random factors (such as power supply voltage fluctuations and friction). Low-frequency gradual variation is the primary characteristic of rotor speed variation.

Based on the characteristics of these error sources, the method for acquiring CT equiangular data of the present disclosure performs calibration and compensation for the above errors, thereby achieving equiangular data acquisition of the CT scanning system.

The present disclosure will be described in further detail below with reference to the accompanying drawings.

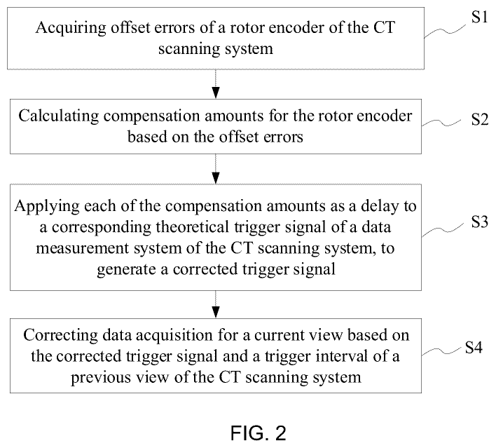

As shown in FIG. 2, in one embodiment, a method for acquiring CT equiangular data of the present disclosure is applied to a CT scanning system and comprises steps S1 to S4.

Step S1 comprises: acquiring offset errors of a rotor encoder of the CT scanning system.

Specifically, acquiring the offset errors of the rotor encoder of the CT scanning system comprises:
11) calculating an offset error Error (i) corresponding to each pulse of the rotor encoder according to Error(i)=AngAct(i)−AngIdeal(i), wherein AngIdeal(i) represents a theoretical angle of an i-th pulse among the pulses, and AngAct(i) represents an actual angle of the i-th pulse.

In one embodiment, it is assumed that a first pulse following an index pulse of the rotor encoder is defined as a zero-angle position of the rotor, and angle calculation is performed based on this reference. It should be noted that any pulse may be selected as the zero-angle position, and such selection does not affect the accuracy of the algorithm.

The total number of pulses generated by the rotor encoder for one full rotation is defined as N. For any pulse i,

$$AngIdeal ( i ) = ( \frac{360}{N} ) * i .$$

12) determining a maximum pulse delay of the rotor encoder according to MaxDelay=Max(Error(1), Error(2), . . . , Error(N)), wherein N represents a total number of pulses generated by the rotor encoder for one full rotation, and 1≤i≤N.
13) calculating the offset errors of the rotor encoder according to ErrorNew(i)=MaxDelay−Error(i)+SafMar, wherein SafMar represents a safety margin.

The offset error may be zero, positive, or negative. Being zero indicates that the actual angular position exactly matches the theoretical angular position. A positive value indicates that the actual pulse arrives later than expected, meaning the actual angular position is greater than the theoretical angular position. A negative value indicates that the actual pulse arrives earlier than expected, meaning the actual angular position is smaller than the theoretical angular position. Using the above offset errors ensures that only pulse delays occur in subsequent compensation operations. Shifting all encoder signals and the origin signal by the same amount does not affect signal acquisition or image reconstruction.

It should be noted that the safety margin SafMar is used to ensure that each subsequent pulse has a delay. SafMar may be set based on data of the rotor encoder and may be any small constant, for example by dividing 360 by N, and then further dividing the result by 4 (i.e., 360/N/4).

Step S2 comprises: calculating compensation amounts for the rotor encoder based on the offset errors.

Specifically, calculating the compensation amounts for the rotor encoder based on the offset errors comprises:
21) acquiring a rotation time RotTime corresponding to one full rotation of a rotor of the CT scanning system.

The offset errors are expressed in angular units and must be converted into the time domain for real-time acquisition control. Therefore, the rotation time for one full rotation of the rotor is first measured and denoted as RotTime. The rotation time may be obtained using an average value, a median value, or similar algorithms based on measurements over multiple rotations.
22) calculating the compensation amounts according to Comp(i)=Round(ErrorNew(i)/(360*RotTime/ClockCycle), 0), wherein ClockCycle represents a counter cycle of the CT scanning system for processing signals of the rotor encoder.

For ease of processing by the processor, time is expressed in processor clock cycles. Round( ) represents a rounding function.

Step S3 comprises: applying each of the compensation amounts as a delay to a corresponding theoretical trigger signal of a data measurement system of the CT scanning system, to generate a corrected trigger signal. That is, there are multiple compensation amounts and multiple trigger signals, and they correspond to each other in a one-to-one manner.

Specifically, for 360° acquisition, assume that L views are required per rotation (L=N, or N/2, or N/3, etc.). This means that a trigger signal of the data measurement system is generated every M pulses of the rotor encoder, where M=N/L. For example, if the rotor encoder generates 4096 pulses per rotation (N=4096) and 1024 views are required per rotation (L=1024), then every 4 pulses (M=4) a trigger signal of the data measurement system is generated. Therefore, when the processor receives the M-th encoder pulse, assuming it is the k-th encoder pulse counted from the zero-angle position, the processor delays Comp(k) clock cycles and then outputs the trigger signal of the data measurement system.

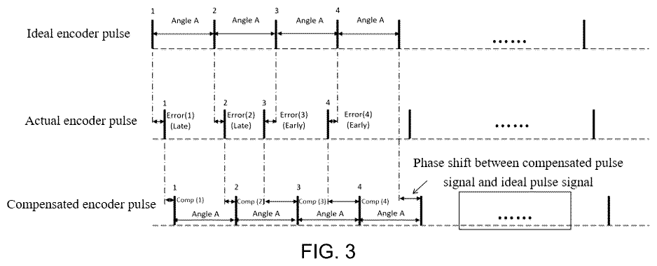

As shown in FIG. 3, the first row illustrates ideal angular pulses for CT equiangular data acquisition. In this case, the angular interval A between any two adjacent pulses is constant. The second row illustrates actual angular pulses, clearly showing errors: pulses 1 and 2 arrive later than the ideal pulses, while pulse 3 and 4 arrive earlier. The angular intervals between adjacent pulses change abruptly. The third row illustrates angular pulses after clock-based compensation. The pulses are shifted in light of the calculated errors, and the angular intervals between the shifted pulses become equal, although the shift amount differs for each pulse. Under a constant rotor speed, the above rotor encoder error calibration and compensation method ensures that trigger signals of the data measurement system are evenly spaced under ideal conditions.

It should be noted that each time an index pulse of the rotor encoder is received, the offset errors and compensation amounts of the rotor encoder of the CT scanning system are reacquired, and the corrected trigger signal is updated accordingly.

Step S4 comprises: correcting data acquisition for a current view based on the corrected trigger signal and a trigger interval of a previous view of the CT scanning system.

Specifically, before CT data acquisition begins, the actual time interval between two adjacent trigger signals (i.e., the trigger interval) is calculated and stored in a register as Int(0). Before each view data acquisition starts, the value of trigger interval Int(0) stored in the register has been updated for each trigger cycle. When the data acquisition process is initiated, the current value of Int(0) is used as the target integration time of the data measurement system.

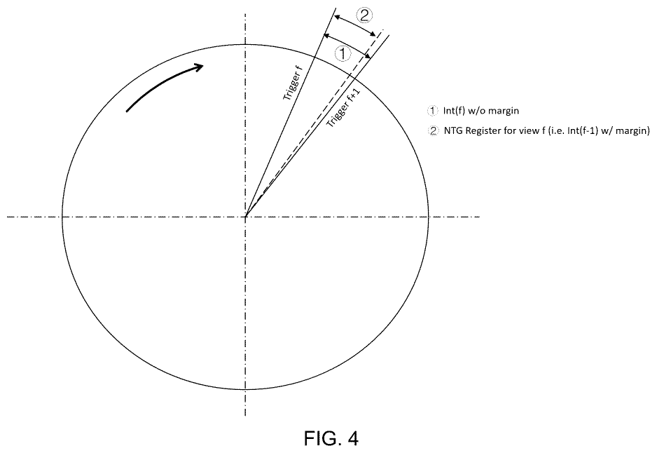

As shown in FIG. 4, for a previous view f (f=1, 2, 3, . . . ), the trigger interval Int (f) of the previous view (f) is obtained by subtracting the actual trigger time of the trigger signal of view (f−1) before the previous view (f) from the actual trigger time of the trigger signal of the previous view (f). The predicted trigger interval IntNew (f) for the current view (f+1) is calculated by subtracting a redundancy amount from the trigger interval Int (f), that is, IntNew(f)=Int(f)−redundancy. The redundancy ensures that integration completes before the next trigger signal arrives, and its value depends on the specific application. IntNew (f) is used as the predicted trigger interval, i.e., the predicted integration time, for view (f+1). Because rotor speed varies only gradually, the integration time varies only slightly from view to view. Finally, the data measurement system performs data acquisition for the current view (f+1) based on the corrected trigger signal and the predicted trigger interval.

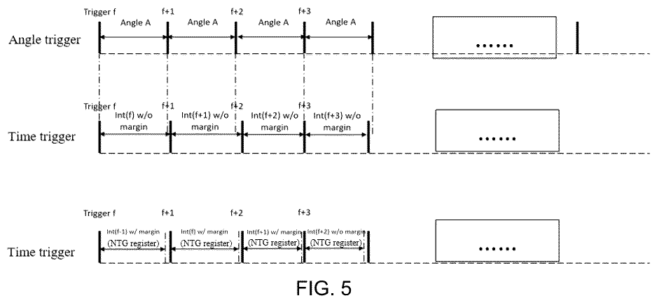

As shown in FIG. 5, the first row illustrates evenly spaced trigger signals in the angular domain. The second row illustrates trigger signals measured in the time domain. Due to variations in rotor speed, signals that are evenly spaced in the angular domain are not evenly spaced in the time domain. The third row illustrates the trigger signals actually used. Because rotor speed can only vary gradually, the actual trigger interval of the previous view is used as the target trigger interval for the next view. The above rotor speed error compensation method ensures that the set trigger interval matches the actual trigger interval. Since the variation in trigger interval is known, the measured data can be corrected so that the integration time is equal across all frames.

The scope of protection of the method for acquiring CT equiangular data described in the present disclosure is not limited to the sequence of operations listed herein. Any scheme realized by adding or subtracting operations or replacing operations of the traditional techniques according to the principle of the present disclosure is included in the scope of protection of the present disclosure.

The present disclosure also provides a system for acquiring CT equiangular data, the system for acquiring CT equiangular data can implement the method for acquiring CT equiangular data described in the present disclosure, but the device for implementing the system for acquiring CT equiangular data described in the present disclosure includes, but is not limited to, the system for acquiring CT equiangular data as described in the present disclosure. Any structural adjustment or replacement of the prior art made according to the principles of the present disclosure is included in the scope of the present disclosure.

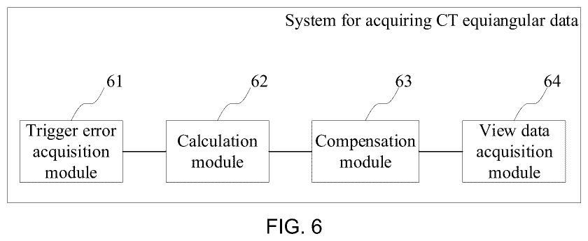

As shown in FIG. 6, in one embodiment, the system for acquiring CT equiangular data of the present disclosure comprises a trigger error acquisition module 61, a calculation module 62, a compensation module 63, and a view data acquisition module 64.

The trigger error acquisition module 61 is configured to acquire offset errors of the rotor encoder of the CT scanning system.

The calculation module 62 is connected to the trigger error acquisition module 61 and is configured to calculate compensation amounts for the rotor encoder based on the offset errors.

The compensation module 63 is connected to the calculation module 62 and is configured to apply each of the compensation amounts as a delay to a corresponding theoretical trigger signal of the data measurement system of the CT scanning system, to generate a corrected trigger signal.

The view data acquisition module 64 is connected to the compensation module 63 and is configured to correct data acquisition for a current view based on the corrected trigger signal and a trigger interval of a previous view of the CT scanning system.

The structures and operating principles of the trigger error acquisition module 61, the calculation module 62, the compensation module 63, and the view data acquisition module 64 correspond respectively to the steps of the method for acquiring CT equiangular data described above.

In the several embodiments proposed in the present disclosure, the disclosed systems, devices, or methods can be implemented in other ways. For example, the embodiments of devices described above are only illustrative, and the division of modules or units is only a division of logical functions. In actual implementation, there may be other division methods, such as multiple modules or units can be combined or integrated into another system, or some features can be ignored or not executed. Here, the coupling or direct coupling or communication connection between each other can be indirect coupling or communication connection through some interfaces, devices, modules, or units, and can be electrical connection, mechanical connection, or other connections.

The modules or units shown as separate components can be physically separated or not. The components shown as modules or units can be physical modules or not. That is, they can be located in one place, or they can also be distributed to multiple network units. Some or all of the modules or units can be selected as needed to achieve the purpose of the embodiment. For example, in one embodiment of the present disclosure, each functional module or unit can be integrated into one processing module. Each functional module or unit can exist physically separately, or two or more modules or units can be integrated into one module or unit.

The ordinary technical personnel in this field should further realize that the units and algorithm steps of each example described in combination with the embodiments disclosed here can be implemented by electronic hardware, computer software, or a combination of both. In the above description, each example's composition and steps have been described generally based on functions, so as to clearly illustrate the interchangeability of hardware and software. Whether these functions are executed by hardware or software depends on the specific application and design constraints of the technical solution. Professional technicians can use different methods to implement the functions described for each specific situation, but such implementation should not be considered beyond the scope of the present disclosure.

The present disclosure further provides a non-transitory computer-readable storage medium. Those skilled in the art can understand that, all or part of the steps in the method for implementing the above embodiments can be implemented when the computer program is executed by a processor. The non-transitory computer-readable storage medium may be, for example, random access memory, read-only memory, flash memory, hard disk, solid-state disk, magnetic tape, floppy disk, optical disc and any combination thereof. The above storage medium can be any available medium that can be accessed by a computer, or a data storage device that integrates one or more available media, such as a server, a data center, etc. The available medium can be a magnetic medium (such as a floppy disk, a hard disk, or a magnetic tape), an optical medium (such as a digital video disc (DVD)), or a semiconductor medium (such as a solid state disk (SSD)), etc.

The present disclosure further provides a CT scanning system. The CT scanning system comprises a processor and a memory.

The memory is configured to store a computer program.

The memory comprises various media capable of storing program code, including Read-Only Memory (ROM), Random Access Memory (RAM), magnetic disks, Universal Serial Bus (USB) flash drives, memory cards, or optical disks.

The processor is connected to the memory and is configured to execute the computer program stored in the memory, so as to cause the CT scanning system to perform the method for acquiring CT equiangular data described above.

Preferably, the processor may be a general-purpose processor, including a Central Processing Unit (CPU), a Network Processor (NP), or the like. The processor may also be a Digital Signal Processor (DSP), an Application Specific Integrated Circuit (ASIC), a Field Programmable Gate Array (FPGA), or other programmable logic devices, discrete gate or transistor logic devices, or discrete hardware components.

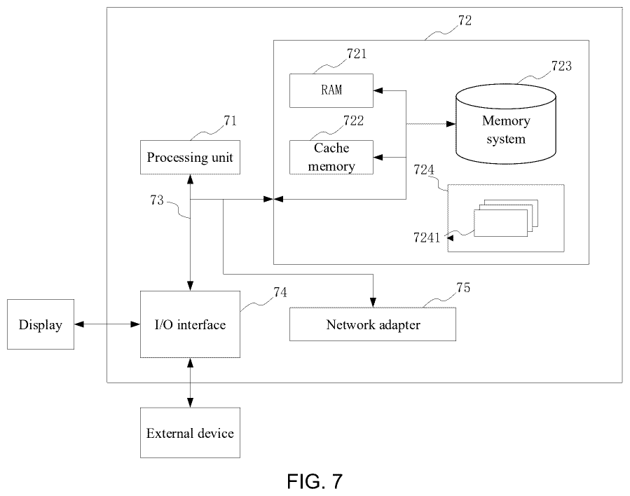

As shown in FIG. 7, the CT scanning system of the present disclosure is embodied in the form of a general-purpose computing device. The components of the CT scanning system may comprise one or more processors or processing units 71, a memory 72, and a bus 73 that connects different system components (including the memory 72 and the processing unit 71).

The bus 73 represents one or more types of bus structures, comprising a memory bus or memory controller, a peripheral bus, a graphics acceleration port, a processor bus, or a local bus using any of a variety of bus architectures. For example, such architectures may comprise Industry Standard Architecture (ISA) bus, Micro Channel Architecture (MAC) bus, Enhanced ISA bus, Video Electronics Standards Association (VESA) local bus, and Peripheral Component Interconnect (PCI) bus.

The CT scanning system typically comprises various computer system readable media. Such media may be any available media accessible by the CT scanning system, including volatile and non-volatile media, removable and non-removable media.

The memory 72 may comprise computer system readable media in the form of volatile memory, such as RAM 721 and/or cache memory 722. The CT scanning system may further comprise other removable/non-removable, volatile/non-volatile computer system storage media. For example, the memory system 723 may be used to read and write to non-removable, non-volatile magnetic media (not shown in FIG. 7, commonly referred to as a “hard disk drive”). Although not shown in FIG. 7, a magnetic disk drive may be provided for reading and writing to removable non-volatile magnetic disks (such as floppy disks), and an optical disk drive may be provided for reading and writing to removable non-volatile optical disks (such as Compact Disc Read-Only Memory (CD-ROM), Digital Versatile Disc Read-Only Memory (DVD-ROM), or other optical media). In such cases, each drive may be connected to the bus 73 by one or more data media interfaces. The memory 72 may comprise at least one program product having a set (for example, at least one) of program modules configured to perform the functions of the embodiments of the present disclosure.

The program/utility 724 having a set (at least one) of program modules 7241 may be stored, for example, in the memory 72. Such program modules 7241 may comprise an operating system, one or more application programs, other program modules, and program data. Each of these examples, or any combination thereof, may implement aspects of a network environment. The program modules 7241 are generally configured to perform the functions and/or methods described in the embodiments of the present disclosure.

The CT scanning system may also communicate with one or more external devices (such as a keyboard, a pointing device, or a display), and may further communicate with one or more devices that enable a user to interact with the CT scanning system, and/or with any devices that enable the CT scanning system to communicate with one or more other computing devices (such as a network interface card, a modem, etc.). Such communication may be performed through an input/output (I/O) interface 74. Additionally, the CT scanning system may communicate with one or more networks (e.g., local area network (LAN), wide area network (WAN), and/or public networks such as the Internet) through a network adapter 75. As shown in FIG. 7, the network adapter 75 communicates with other modules of the CT scanning system through the bus 73. Although not shown in FIG. 7, it should be understood that other hardware and/or software modules can be used in conjunction with the CT scanning system, including but not limited to microcode, device drivers, redundant processors, external disk drive arrays, Redundant Array of Independent Disk (RAID) systems, tape drives, and data backup storage systems.

The above-mentioned embodiments are for exemplarily describing the principle and effects of the present disclosure instead of limiting the present disclosure. Those skilled in the art can make modifications or changes to the above-mentioned embodiments without going against the spirit and the range of the present disclosure. Therefore, all equivalent modifications or changes made by those who have common knowledge in the art without departing from the spirit and technical concept disclosed by the present disclosure shall be still covered by the scope of the present disclosure.

1. A method for acquiring CT equiangular data, applied to a CT scanning system, comprising:
acquiring offset errors of a rotor encoder of the CT scanning system;
calculating compensation amounts for the rotor encoder based on the offset errors;
applying each of the compensation amounts as a delay to a corresponding theoretical trigger signal of a data measurement system of the CT scanning system, to generate a corrected trigger signal; and
correcting data acquisition for a current view based on the corrected trigger signal and a trigger interval of a previous view of the CT scanning system.

2. The method for acquiring CT equiangular data according to claim 1, wherein acquiring the offset errors of the rotor encoder of the CT scanning system comprises:
calculating an offset error Error(i) corresponding to each pulse of the rotor encoder according to Error(i)=AngAct(i)−AngIdeal(i), wherein AngIdeal(i) represents a theoretical angle of an i-th pulse among the pulses, and AngAct(i) represents an actual angle of the i-th pulse;
determining a maximum pulse delay of the rotor encoder according to MaxDelay=Max(Error(1),Error(2), . . . ,Error(N)), wherein N represents a total number of pulses generated by the rotor encoder for one full rotation, and 1≤i≤N; and
calculating the offset errors of the rotor encoder according to ErrorNew(i)=MaxDelay−Error(i)+SafMar, wherein SafMar represents a safety margin.

3. The method for acquiring CT equiangular data according to claim 2, wherein
$$AngIdeal ( i ) = ( \frac{360}{N} ) * i .$$

4. The method for acquiring CT equiangular data according to claim 2, wherein calculating the compensation amounts for the rotor encoder based on the offset errors comprises:
acquiring a rotation time RotTime corresponding to one full rotation of a rotor of the CT scanning system; and
calculating the compensation amounts according to Comp(i)=Round(ErrorNew(i)/(360*RotTime/ClockCycle), 0), wherein ClockCycle represents a counter cycle of the CT scanning system for processing signals of the rotor encoder.

5. The method for acquiring CT equiangular data according to claim 1, wherein a first pulse following an index pulse of the rotor encoder is configured as a zero-angle position of a rotor of the CT scanning system.

6. The method for acquiring CT equiangular data according to claim 1, wherein the method further comprises: each time after receiving an index pulse of the rotor encoder, reacquiring the offset errors of the rotor encoder of the CT scanning system to update the corrected trigger signal.

7. The method for acquiring CT equiangular data according to claim 1, wherein correcting the data acquisition for the current view based on the corrected trigger signal and the trigger interval of the previous view of the CT scanning system comprises:
acquiring the trigger interval Int (f) of the previous view (f) by subtracting an actual trigger time of a trigger signal of view (f−1) before the previous view (f) from an actual trigger time of a trigger signal of the previous view (f);
calculating a predicted trigger interval IntNew (f) for the current view (f+1) by subtracting a redundancy amount and/or an integration-termination advance amount from the trigger interval Int (f); and
performing the data acquisition for the current view (f+1) based on the corrected trigger signal and the predicted trigger interval.

8. A system for acquiring CT equiangular data, applied to a CT scanning system, comprising a trigger error acquisition module, a calculation module, a compensation module, and a view data acquisition module, wherein:
the trigger error acquisition module is configured to acquire offset errors of a rotor encoder of the CT scanning system;
the calculation module is configured to calculate compensation amounts for the rotor encoder based on the offset errors;
the compensation module is configured to apply each of the compensation amounts as a delay to a corresponding theoretical trigger signal of a data measurement system of the CT scanning system, to generate a corrected trigger signal; and
the view data acquisition module is configured to correct data acquisition for a current view based on the corrected trigger signal and a trigger interval of a previous view of the CT scanning system.

9. A CT scanning system, comprising a processor and a memory, wherein:
the memory is configured to store a computer program; and
the processor is configured to execute the computer program stored in the memory, to cause the CT scanning system to perform the method for acquiring CT equiangular data according to claim 1.

10. A non-transitory computer-readable storage medium, which stores a computer program, wherein the method for acquiring CT equiangular data according to claim 1 is implemented when the computer program is executed by a processor.
